(19) United States
(12) Patent Application Publication
(10) Pub. No.: US 2026/0232308 A1
(43) Pub. Date: Aug. 13, 2026

(54) Knotless Constructs and Methods of Tissue Repair

(71) Applicant: Arthrex, Inc., Naples, FL (US)

(72) Inventors: Shaun G. LEBLANC, Naples, FL (US); Mark D. FEENEY, Naples, FL (US); Brittany R. FOSLIEN, Naples, FL (US); Jason A. VALENTIN, Fort Myers, FL (US)

(73) Assignee: Arthrex, Inc., Naples, FL (US)

(21) Appl. No.: 19/536,510

(22) Filed: Feb. 11, 2026

Related U.S. Application Data
(60) Provisional application No. 63/757,379, filed on Feb. 12, 2025.

(51) Int. Cl. A61B 17/04

(52) U.S. Cl. CPC A61B 17/0401; A61B 2017/0409; A61B 2017/0414; A61B 2017/0464

(57) ABSTRACT

Tensionable, knotless, self-locking surgical constructs and methods for surgical repairs are disclosed. A surgical repair system includes two or more soft anchors. The soft anchors may be all-suture anchors that are interconnected by a flexible coupler extending from one suture anchor to another suture anchor.

### CROSS-REFERENCE TO RELATED APPLICATIONS

The application claims priority to U.S. Provisional Patent Application No. 63/757,379 filed Feb. 12, 2025, the disclosure of which is hereby incorporated be reference in its entirety herein.

### BACKGROUND

The disclosure relates to the field of surgery and, more specifically, to knotless anchor constructs and associated methods of tissue repairs.

### SUMMARY

Reconstruction systems, assemblies and methods for fixation of tissue are disclosed.

A surgical construct may create a knotless, self-locking, reinforced repair. A surgical construct may be made completely of suture to achieve fixation in bone without a separate anchoring body. A surgical construct may be employed in knotless fixation of first tissue to second tissue, for example, fixation of soft tissue to bone. A surgical construct may be a knotless, tensionable construct that includes two or more soft anchors. In some implementations, the soft anchors may be all-suture anchors. In some implementations, at least one of the soft anchors may include a soft anchor sheath with one or more repair sutures. In some implementations, at least one of the repairs sutures may be connected to another soft anchor.

Methods of tissue repairs are also disclosed. In some implementations, knotless surgical constructs provide tissue fixation without any knot formation, by providing an all-suture repair which is conducted with fewer steps, increased suture management, and increased reproducibility.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a soft anchor.

FIG. 2 illustrates another soft anchor.

FIG. 3 illustrates the soft anchor of FIG. 2 with an exemplary shuttle link.

FIG. 4 illustrates the soft anchor of FIG. 3 on an inserter.

FIG. 5 illustrates a schematic surgical repair with the soft anchors of FIGS. 1 and 2.

FIGS. 6-8 illustrate subsequent steps of the surgical repair of FIG. 5.

FIG. 9 illustrates another soft anchor.

FIG. 10 illustrates the soft anchor of FIG. 9 on an inserter.

FIG. 11 illustrates a schematic surgical repair with the soft anchors of FIGS. 1 and 9.

FIGS. 12-14 illustrate subsequent steps of the surgical repair of FIG. 11.

FIG. 15 illustrates another surgical repair.

FIG. 16 illustrates another surgical repair.

FIG. 17 illustrates another surgical repair.

FIG. 18 illustrates another surgical repair.

### DETAILED DESCRIPTION

Tensionable, knotless, self-locking surgical constructs can create knotless, reinforced repairs.

A soft tissue repair system may include a knotless, tensionable construct. In some implementations, a knotless, tensionable construct may include two or more soft anchors. In some implementations, a knotless, tensionable construct may consist of two all-suture anchors. A knotless, tensionable construct may consist of two all-suture anchors that are connected to each other in a knotless manner.

In some implementations, a knotless, tensionable construct may include (i) a soft suture anchor and (ii) a static anchor with a fixed loop. The soft suture anchor may be connected to the fixed loop of the static anchor. The soft suture anchor may include one or more repair sutures (flexible strands) attached to an anchor sheath. The static anchor may include a fixed loop attached to an anchor sheath. The fixed loop may be formed of round suture. The fixed loop may be formed of tape/flat suture. The fixed loop may be formed of bifurcated or multiple-furcated suture. In some implementations, the round suture may be provided with no core to allow the fixed loop to lay flat on tissue. In some implementations, the fixed loop may slide relative to a sheath of the static anchor. In some implementations, the fixed loop may slide freely relative within the sheath of the static anchor. In some implementations, the fixed loop may not slide but be fixed within the sheath of the static anchor. The soft suture anchor may include one or more shuttle/pull devices. In some implementations, the knotless, tensionable construct may form a knotless, tensionable InternalBrace™ construct. In some implementations, the knotless, tensionable construct may form a SpeedBridge™ type construct that includes a plurality of connected soft anchors.

In some implementations, a knotless, tensionable construct may include (i) a soft suture anchor and (ii) an anchor sheath. The soft suture anchor may include one or more repair sutures (flexible strands) attached to a sheath. The soft suture anchor may be connected to the anchor sheath by at least one of the repair sutures (flexible strands). The soft suture anchor may include one or more shuttle/pull devices.

The surgical constructs may include a plurality of soft anchors that may be employed by themselves or in combination with additional hard-body anchors such as SwiveLock® anchors and/or PushLock® anchors, among others.

Methods of knotless tissue repairs are also disclosed. In some implementations, a surgical construct provides knotless first tissue to second tissue fixation, for example, knotless fixation of soft tissue (ligament, tendon, graft, etc.) to bone without any knot formation and in a simple and fast manner. The tensionable repairs with all-suture anchors facilitate drill tunnel holes smaller than those of traditional techniques that utilize hard body anchors and require larger bone holes.

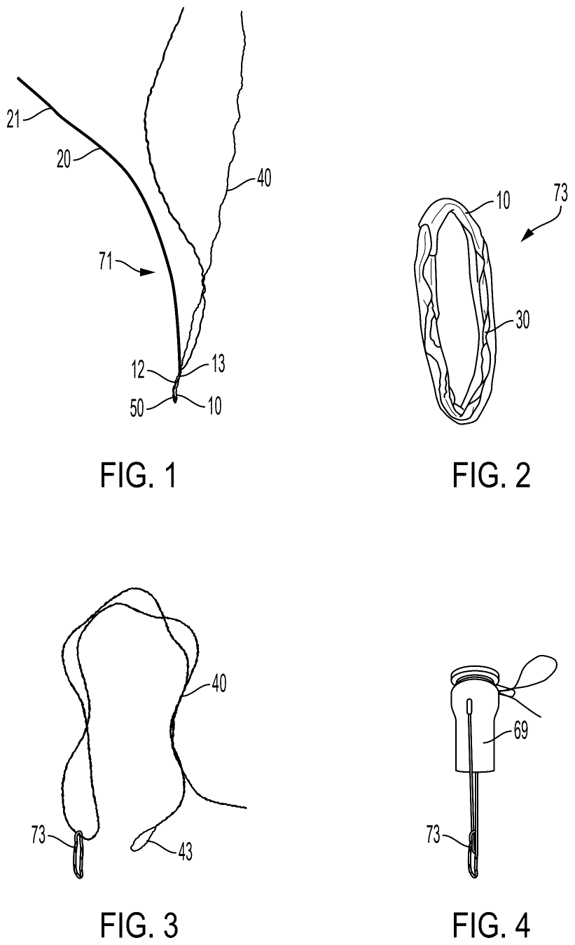

Referring now to the drawings, where like elements are designated by like reference numerals, FIGS. 1-18 illustrate exemplary repairs 101, 201, 101a, 101b, 201a, 201b as well as various structural elements of exemplary surgical repair systems 100, 200. Repair system 100, 200 may be a surgical assembly. Repair system 100, 200 may be a tensionable, knotless construct. Repair system 100, 200 may be an implantable device. Repair system 100, 200 may be a tensionable, knotless, self-locking, surgical assembly. FIGS. 7, 8 and 13-18 illustrate exemplary soft tissue repairs 101, 101a, 101b, 201, 201a, 201b with surgical constructs 100, 200.

As detailed below, surgical constructs 100, 200 are formed of implantable devices made completely of suture or sutures that achieve fixation in bone without a separate anchoring body. The design enables for fixation by deployment into bone with one or more suture tails remaining outside the bone for device attachment, tensioning and/or alternative usage.

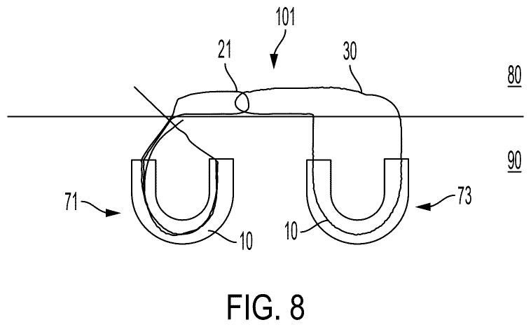

FIG. 1 illustrates a soft suture anchor 71. FIG. 2 illustrates a static anchor 73 with a fixed loop 30. Repair system 100 of FIGS. 7 and 8 includes (i) soft suture anchor 71 and (ii) static anchor 73 with a fixed loop 30. The soft suture anchor 71 is connected to the fixed loop 30 of the static anchor 73.

The soft suture anchor 71 of FIG. 1 may include one or more repair sutures 20 (flexible strands or couplers 20) attached to fixation device 10. As shown in FIG. 1, fixation device 10 can be in the form of a soft anchor sleeve 10 (anchor sheath, suture tube or tubular member 10) with a cannulation 11 (not shown) with two ends 12, 13. At least one of the two ends 12, 13 may be an open end. Flexible coupler 20 extends through cannulation 11 of sheath 10 and comes out of sheath 10. In some implementations, flexible coupler 20 may include a single flexible end 21. Flexible coupler 20 forms at least one flexible, closed, knotless, continuous loop 50. Flexible coupler 20 may be in the form of round, tape or bifurcated suture. Flexible coupler 20 may be a flexible strand, a suture, a repair suture. The anchor sheath 10 may be of any desired length and diameter and may have a tail, two tails, or no tails.

FIG. 1 also illustrates a shuttle/pull device 40 extending through the sleeve 10. Shuttle/pull device 40 may be a suture passing instrument, a suture passer, a shuttle link, a FiberLink™, a nitinol loop. As detailed below, device 40 includes an eyelet/loop 43 for passing flexible coupler 20. If more than one shuttle/pull devices 40 are provided, the shuttle/pull devices can extend through the sheath 10 in similar or different directions and/or orientations and/or locations. Details of an exemplary soft suture anchor with a soft anchor sleeve (sheath or tubular member) and flexible shuttling strands are set forth, for example, in U.S. application Ser. No. 15/998,516 entitled “Methods of Tissue Repairs” filed on Aug. 16, 2018 (issued as U.S. Pat. No. 10,849,734 on Dec. 1, 2020), the disclosure of which is incorporated by reference in its entirety herein. The flexible coupler and the one or more shuttle/pull devices can extend through the sleeve in similar or different directions and/or orientations and/or locations.

Flexible coupler 20 of FIG. 1 is provided with a single terminal end 21. The terminal end 21 is a flexible end (free end 21) that may be provided integral with sheath 10. Shuttle/pull device 40 is passed through the sheath 10. Shuttle/pull device 40 may be attached to the sheath 10 and/or flexible coupler 20 by being spliced, for example, to the flexible coupler. The flexible coupler 20 can be passed through at least a portion of the body of the sheath 10, for example, through a full length of the sheath, or may enter and/or exit the body of the sheath at a location other than most distal end and most proximal end of sheath 10. The sheath 10 may have any desired length and diameter and may have one or more tails or no tail.

In some implementations, soft suture anchor 71 may be a knotless FiberTak® soft anchor that may be single-loaded or multiple-loaded.

FIG. 2 illustrates static anchor 73 which includes fixation device 10 and loop 30. Fixation device 10 may be similar to the soft anchor sleeve 10 (sheath or tubular member 10) of soft suture anchor 71 of FIG. 1. Loop 30 extends through cannulation 11 of sheath 10 and comes out of sheath 10. Sheath 10 may be pre-loaded with loop 30. Sheath 10 may have any desired length and diameter and may have one or two tails, or no tail (flap(s) or no flap).

In some implementations, loop 30 may be a fixed suture loop at a set length. In some implementations, loop 30 may be formed of round suture. The round suture allows easier splicing and formation of a round loop. The splicing may be located within sheath 10 and at a couple locations. In some implementations, the round suture may be provided with no core to allow the fixed loop 30 to lay flat on tissue. In some implementations, the fixed loop 30 may slide freely within to sheath 10 of the static anchor 73. In some implementations, the fixed loop 30 may not slide relative to sheath 10 and may be fixed to the sheath 10 of the static anchor 73. The fixed loop 30 may be stitched to the sheath 10 or weaved in and out of the sheath 10. The length and diameter of loop 30 may vary and depend on the specific indication and tissue anatomy.

Loop 30 may be a continuous, flexible loop having a fixed diameter and length. In some implementations, loop 30 may be formed by splicing a round suture to form a round loop with a round cross-section. The splice(s) may be located within sheath 10 of suture anchor 73.

In some implementations, and as shown in FIG. 3, the static anchor 73 may include a shuttle/pull device 40 or a strand of suture (round or flat/tape) to aid in passing of the flexible coupler 20 (repair suture 20) through the fixed loop 30, as detailed below. FIG. 4 illustrates the static anchor 73 with shuttle/pull device 40 secured on inserter 69. Although the implementations below will be described with reference to a shuttle/pull device with a loop, such as shuttle/pull device 40 with loop 43, the disclosure is not limited to this exemplary only device and also contemplates a simple suture strand (round or flat/tape) to aid in passing of the flexible coupler 20.

In some implementations, static anchor 73 may be a FiberTak® loop anchor. The static anchor 73 may be inserted unicortically or bicortically. In some implementations, static anchor 73 may be formed as a unitary, integral construct. In some implementations, static anchor 73 may be a one-piece machined construct.

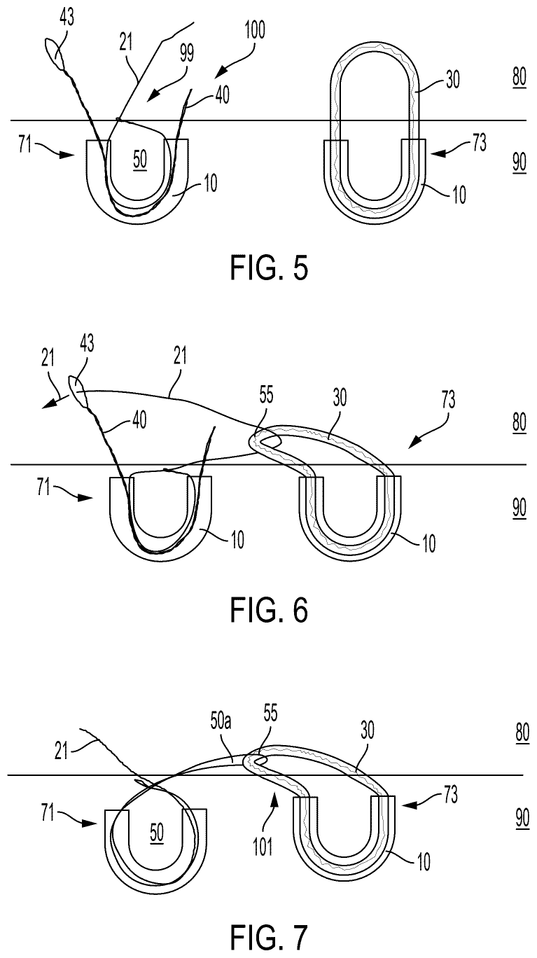

Reference is now made to FIGS. 5-8 which depict steps of tissue repair 101 with soft anchors 71, 73 of system 100. Fixed loop 30 of static anchor 73 is connected to knotless mechanism 99 of soft anchor 71 as part of repair 101 that attaches tissue 80 to another tissue 90. In some implementations, the repair suture 20 may pass through tissue 80. In some implementations, the repair suture 20 may just sit over tissue 80, without passing through it. Although the implementations below will be described with reference to repair suture 20 passing through or around the tissue 80, the disclosure is not limited to this exemplary-only implementation and contemplates the repair suture not passing through or around tissue, but just sitting over the top of tissue 80.

FIG. 5 illustrates soft anchors 71, 73 of repair system 100 inserted within a first tissue 90 (for example, hard tissue such as bone). Soft anchors 71, 73 may be inserted into first tissue 90 either unicortically or bicortically.

FIG. 6 shows end 21 of repair suture 20 from soft anchor 71 passed through, around, or over soft tissue 80, through loop 30 of soft anchor 73, and through eyelet 43 of shuttle/pull device 40 to allow end 21 of repair suture 20 to go back through the sheath 10 of soft anchor 71. Pulling the shuttle/pull device 40 out of sheath 10 allows the repair suture 20 to be pulled towards the body of soft anchor 71, inside the sheath 10, and then exiting the sheath 10 to form another knotless, tensionable loop 50a around, through, or over soft tissue 80. Loop 50a may be a closed loop. Loop 50a may be a continuous, uninterrupted loop. Loop 50a may be an adjustable loop. Passage of repair suture 20 through loop 30 of soft anchor 73 and back through the lumen of the sheath 10 also forms loop interconnection 55, locking the construct. Fixed loop 30 of suture anchor 73 is connected to knotless mechanism 99 of suture anchor 71. The suture end 21 of repair suture 20 can be tensioned and cut. Alternatively, in some implementations, suture end 21 can be used for other tissue repairs that can include knotted repairs, knotless repairs, lateral tissue compression conducted with accompanying interference anchors, etc.

FIGS. 7 and 8 illustrate schematic views of tissue repair 101 that allows tissue 80 to be attached to tissue 90 with repair system 100. The all-suture anchor repair 101 is a tensionable, knotless repair that facilitates smaller drill tunnel holes, fewer surgical steps, and increased suture management during the repair.

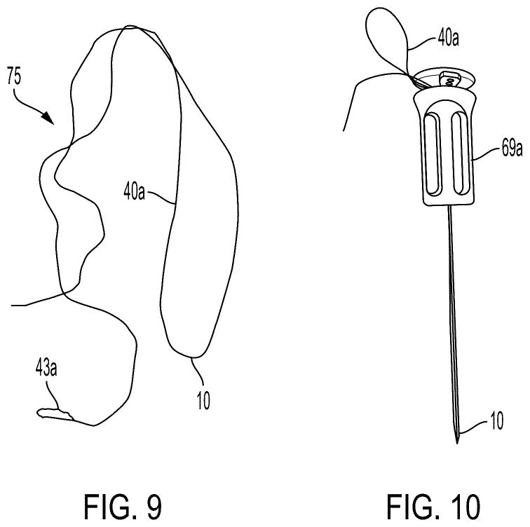

Reference is now made to FIGS. 9 and 10 which illustrate a soft suture anchor 75 provided with a soft anchor sleeve 10 that may be similar to the soft anchor sleeve 10 (anchor sheath or tubular member 10) of suture anchors 71 and 73. FIG. 9 illustrates soft suture anchor 75 attached to a shuttle/pull device 40a with eyelet 43a. FIG. 10 illustrates soft suture anchor 75 with shuttle/pull device 40a secured on inserter 69a. In some implementations, soft suture anchor 75 may be a FiberTak® Button construct.

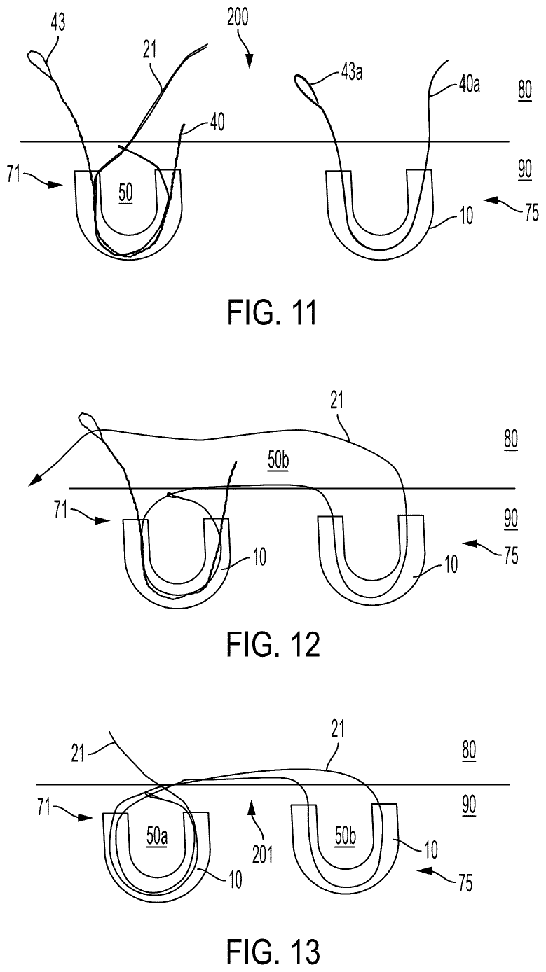

FIGS. 11-14 depict steps of tissue repair 201 with soft anchors 71, 75 of system 200. Sheath 10 of soft anchor 75 is connected to knotless mechanism 99 of soft anchor 71 as part of repair 201 for attaching tissue 80 to another tissue 90.

FIG. 11 illustrates soft anchors 71, 75 of repair system 200 inserted within a first tissue 90 (for example, hard tissue such as bone). Soft anchors 71, 75 may be inserted into first tissue 90 either unicortically or bicortically. Soft anchor 75 may be provided with a shuttle/pull device 40a having eyelet 43a to aid in passing of end 21 of repair suture 20.

FIG. 12 shows end 21 of repair suture 20 from soft anchor 71 passed through, around, or over soft tissue 80, through the sheath 10 of anchor 75, and through eyelet 43 of shuttle/pull device 40 to allow end 21 of repair suture 20 to go back through the sheath 10 of soft anchor 71. Pulling the shuttle/pull device 40 out of sheath 10 of anchor 71 allows the repair suture 20 to pass again through the lumen of the sheath 10 and to form another knotless, tensionable loop 50a, along with additional loop 50b, locking the construct. Sheath 10 of suture anchor 75 is connected to knotless mechanism 99 of suture anchor 71 through loop 50b. Loop 50b may be a continuous, uninterrupted loop. Loop 50b may be a flexible, adjustable loop. Loop 50b passes through or around tissue 80.

As noted above, passage of end 21 of repair suture 20 through sheath 10 of anchor 75 may be facilitated by passing end 21 through eyelet 43a of shuttle/pull device 40a and then pulling out the shuttle/pull device 40a from the sheath 10 of soft anchor 75. End 21 of repair suture 20 passes therefore from one end of sheath 10 of anchor 75 to another end of sheath 10 of soft anchor 75, before being passed back through sheath 10 of soft anchor 71. Subsequent to the tensioning of the repair, the suture end 21 of flexible coupler 20 can be tensioned and cut. In some implementations, suture leg 21 can be used for other tissue repairs that can include knotted repairs, knotless repairs, lateral tissue compression conducted with accompanying interference anchors, etc.

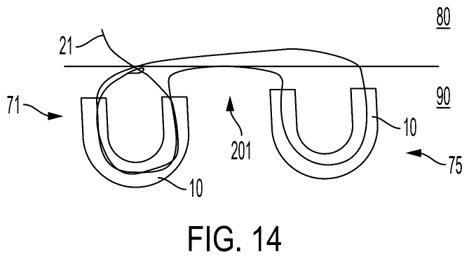

FIGS. 13 and 14 illustrate schematic views of tissue repair 201 that attaches tissue 80 to issue 90 with repair system 200. The all-suture anchor repair 201 is a tensionable, knotless repair that facilitates smaller drill tunnel holes, fewer surgical steps, and increased suture management during the repair.

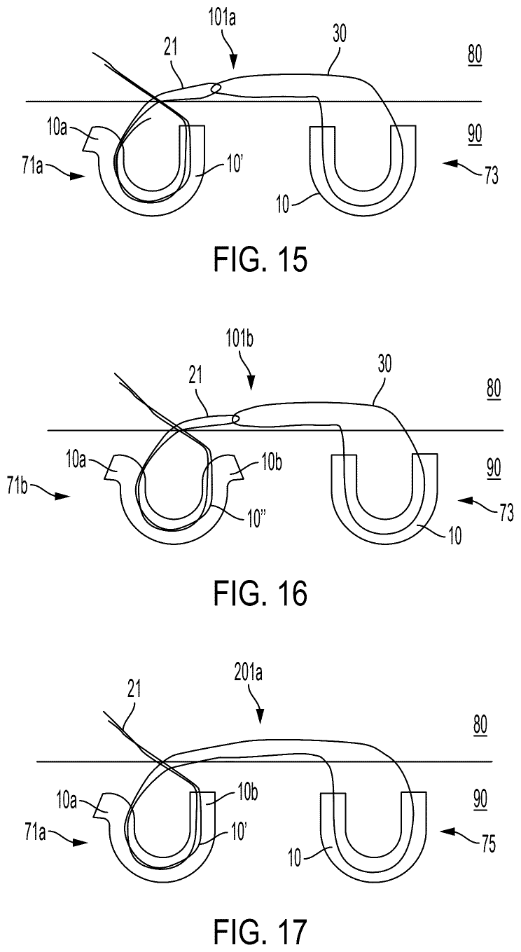

Reference is now made to FIGS. 15-18 which illustrate exemplary tissue repairs 101a, 101b, 201a, 201b that also allow for attachment of tissue 80 to tissue 90.

Repairs 101a, 101b (FIGS. 15 and 16) are about similar to repair 101 of FIG. 8 in that they also employ a static suture anchor 73 provided with a closed loop 30. However, soft suture anchor 71a of FIG. 15 includes a soft anchor sleeve 10′ (anchor sheath, suture tube or tubular member 10′) having one tail or flap 10a. Like in the implementation of FIG. 8, the one or more repair sutures 20 (flexible strands or couplers 20) attaches to fixation device 10′ but passes through only a length of the cannulation of the sleeve 10′, i.e., not through the tail or flap 10a. The repair suture 20 does not pass through full length of the sheath 10′, and enters and/or exits the body of the sheath at a location other than most distal end and most proximal end of sheath 10′. Similarly, loop 30 may also enter and/or exit the body of the sheath 10 at a location other than most distal end and most proximal end of sheath 10 of static anchor 73.

Soft suture anchor 71b of repair 101b of FIG. 16 includes a soft anchor sleeve 10″ (anchor sheath, suture tube or tubular member 10″) having two tails or flaps 10a, 10b. Tails 10a, 10b may have similar or different dimensions. Like in the implementation of FIG. 8, the one or more repair sutures 20 (flexible strands or couplers 20) attaches to fixation device 10″ but does not pass through the full length of the cannulation of the sleeve 10″, i.e., does not pass through the tails or flaps 10a, 10b. The repair suture 20 enters and/or exits the body of the sheath 10″ at locations spaced apart from than most distal end and most proximal end of sheath 10″. Similarly, loop 30 of static anchor 73 may also enter and/or exit the body of the anchor sheath 10 at a location other than most distal end and most proximal end of sheath 10 of static anchor 73.

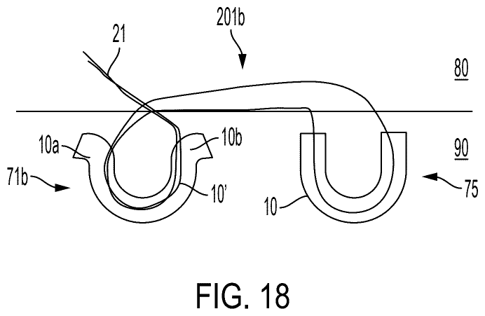

Repairs 201a, 201b (FIGS. 17 and 18) are about similar to repair 201 of FIG. 14 in that they also employ a suture anchor 75 which attaches to a soft anchor. However, soft suture anchor 71a of FIG. 17 includes a soft anchor sleeve 10′ (anchor sheath, suture tube or tubular member 10′) having one tail or flap 10a. Like in the implementation of FIG. 14, the one or more repair sutures 20 (flexible strands or couplers 20) attaches to fixation device 10′ but does not pass through a full length of the cannulation of the sleeve 10′, i.e., does not pass through the tail or flap 10a.

Soft suture anchor 71b of repair 201b of FIG. 18 includes a soft anchor sleeve 10″ (anchor sheath, suture tube or tubular member 10″) having two tails or flaps 10a, 10b. Tails 10a, 10b may have similar or different dimensions. Like in the implementation of FIG. 14, the one or more repair sutures 20 (flexible strands or couplers 20) attaches to fixation device 10″ but does not pass through the full length of the cannulation of the sleeve 10″, i.e., does not pass through the tails or flaps 10a, 10b.

Although the embodiments above have been described with reference to repair systems with only two exemplary soft anchors that are interconnected by a knotless mechanism, it must be understood that the disclosure is not limited to these exemplary-only embodiments. Accordingly, the disclosure also contemplates any number of soft anchors (such as soft anchors 71, 71a, 71b, 73, 75) and in any combination, that are interconnected to confer the advantages of the present disclosure. Any of the soft anchors detailed above may be also provided with a plurality of repair sutures 20 (flexible couplers 20) and/or a plurality of shuttle/pull devices 40, 40a.

If more than one flexible coupler 20 and shuttle/pull device 40, 40a are employed, the plurality of flexible couplers and shuttle/pull devices can reside within the sheath 10, 10′, 10″ independent from each other. The flexible couplers may be similar to each other or different from each other. The shuttle/pull devices may be similar to each other or different from each other. In some implementations, the flexible coupler can be bifurcated or furcated from the sheath 10, 10′, 10″ to provide a plurality of loops.

In some implementations, the flexible coupler 20 may come out of the cannulated sheath 10, 10′, 10″ and be braided to a smaller diameter round suture 21 which may be passed back through the sheath. The braided suture may be furcated/branched/forked (for example, bifurcated or trifurcated, etc.) to provide a plurality of passing sutures.

In some implementations, end 21 of flexible coupler 20 may be a tapered end. In some implementations, the diameters of end 21 may be smaller than the diameters of flexible coupler 20. In some implementations, flexible coupler 20 may be a single strand suture. In some implementations, flexible coupler 20 may be a single braided suture.

Although the implementations above have been detailed with reference to soft tissue 80 attached to bone 90, it must be understood that the disclosure is not limited to these exemplary-only embodiments. Accordingly, the disclosure also contemplates embodiments wherein the knotless, tensionable constructs detailed above may provide bone-to-bone fixation such as repairs associated with patellar fractures, for example, and/or tissue repairs within bone 90 only (without soft tissue 80).

The constructs, systems, and assemblies of the present disclosure may be employed in numerous knotless soft tissue repairs and fixations, for example, fixation of soft tissue to bone. In some implementations, a surgical construct 100, 200 may provide first tissue to second tissue fixation, for example, fixation of soft tissue 80 (ligament, tendon, graft, etc.) to bone 90 in a simple and fast manner.

A knotless, tensionable construct 100 may include (i) a soft suture anchor 71, 71a, 71b and (ii) a static anchor 73 with a fixed loop 30. The soft suture anchor 71, 71a, 71b may be connected to the fixed loop 30 of the static anchor 73 by a knotless mechanism 99. The soft suture anchor 71, 71a, 71b may include one or more repair sutures 20 (flexible strands 20) attached to an anchor sheath 10, 10′, 10″. The static anchor 73 may include a fixed loop 30 attached to an anchor sheath 10. The fixed loop 30 may be formed of round suture. The fixed loop 30 may be formed of tape/flat suture. The fixed loop 30 may be formed of bifurcated suture. In some implementations, the round suture may be provided with no core to allow the fixed loop 30 to lay flat on tissue 90. In some implementations, the fixed loop 30 may slide relative to a sheath 10 of the static anchor 73. In some implementations, the fixed loop 30 may be affixed to (or fixed to) a sheath 10 of the static anchor 73 (may not slide within the sheath). The sheath of the static anchor may have one or more flaps (tails) or may not have any flaps or tails. The soft suture anchor 71, 71a, 71b may include one or more shuttle/pull devices 40 or suture strands (round or flat/tape or furcated). The knotless, tensionable construct 100 may be a knotless, tensionable InternalBrace™ construct.

A knotless, tensionable construct 200 may include (i) a soft suture anchor 71, 71a, 71b and (ii) an anchor sheath 10. The soft suture anchor 71, 71a, 71b may include one or more repair sutures 20 (flexible strands 20) attached to a sheath 10, 10′, 10″. The soft suture anchor 71, 71a, 71b may be connected to the anchor sheath 10 by at least one of the repair sutures 20. The soft suture anchor 71, 71a, 71b may include one or more shuttle/pull devices 40.

Methods of soft tissue repair which do not require tying of knots and allow adjustment of both the tension of the suture and the location of the tissue with respect to the bone, while providing a self-locking mechanism, are disclosed.

A method of knotless tissue repair 101, 101a, 101b comprises inter alia the steps of: securing a plurality of suture anchors 71, 71a, 71b, 73 into a first tissue 90; and connecting at least one flexible coupler 20 attached to one of the suture anchors 71, 71a, 71b to another suture anchor 73. The method may further include the steps of: passing the flexible coupler 20 of suture anchor 71, 71a, 71b around, through, or over a second tissue 80 to be positioned relative to the first tissue 90 and then through a loop 30 of suture anchor 73; and, subsequently, passing the flexible coupler back through sheath 10, 10′, 10″ of suture anchor 71, 71a, 71b by employing a shuttle/pull device 40 to form a loop interconnection 55 between the suture anchors 71, 71a, 71b, 73 and an adjustable, knotless, closed, continuous loop 50a around, through, or over the second tissue 80. Loop 50a can have an adjustable perimeter.

A method of knotless tissue repair 201, 201a, 201b comprises inter alia the steps of: securing a plurality of suture anchors 71, 71a, 71b, 75 into a first tissue 90; and connecting at least one flexible coupler 20 attached to one of the suture anchors 71, 71a, 71b to another suture anchor 75. The method may further include the steps of: passing the flexible coupler 20 of suture anchor 71, 71a, 71b around, through, or over a second tissue 80 to be positioned relative to the first tissue 90 and then through a sheath 10 of suture anchor 75; and, subsequently, passing the flexible coupler 20 back through sheath 10, 10′, 10″ of suture anchor 71, 71a, 71b by employing a shuttle/pull device 40 to form an adjustable, knotless, closed, continuous loop 50b around, through, or over the second tissue 80. Loop 50b can have an adjustable perimeter.

As detailed above, when the anchor sheath 10, 10′, 10″ of all-suture anchors 71, 71a, 71b, 73 is inserted into tissue 90, the anchor sheath has at least one repair suture limb 21 (which is fixed to the anchor and manufactured at the same time with the anchor, as a one-piece construct) and also one or more shuttle links 40. The anchor sheath 10, 10′, 10″ resides within the bone 90. The repair suture limb(s) 21 resides on top of the bone 90. The repair suture 21 is passed around, through, or over the soft tissue 80, and then shuttled through a loop or through another anchor sheath of another all-suture anchor 73, 75. The steps can be repeated if additional links are present (with a single suture limb) and/or if additional suture limbs and additional links are present.

The sheath 10, 10′, 10″ and repair suture 20 can include any flexible material, for example, multifilament, braided, knitted, woven suture, or can include fibers of ultrahigh molecular weight polyethylene (UHMWPE) or the FiberWire® suture (disclosed in U.S. Pat. No. 6,716,234, the disclosure of which is hereby incorporated by reference in its entirety herein).

Repair suture 20 may be any flexible coupler or flexible strand and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Repair suture 20 may have cross-sections of various forms and geometries, including round, oval, rectangular, or flat, among others, or combination of such forms and geometries.

In some implementations and as noted above, repair suture 20 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Repair suture 20 may be braided or multi-filament suture such as FiberTape® suture tape (as disclosed in U.S. Pat. No. 7,892,256, the disclosure of which is incorporated in its entirety herewith). Repair suture 20 may include elastic material. Repair suture 20 may consist essentially of elastic suture.

Repair suture 20 can be also formed of a stiff material, or combination of stiff and flexible materials, particularly for the regions of the coupler that may need to be passed/spliced through the body of the coupler and depending on whether they are employed with additional fixation devices.

Various structural elements of surgical construct 100, 200 may be visually coded, making identification and handling of the sheath and suture legs simpler. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A tissue repair system, comprising a first soft anchor knotlessly connected to a second soft anchor by a flexible coupler, wherein at least one of the first soft anchor and the second soft anchor is an all-suture soft anchor.

2. The tissue repair system of claim 1, wherein the first soft anchor includes a soft anchor sheath and the flexible coupler extends through at least a portion of the soft anchor sheath.

3. The tissue repair system of claim 2, wherein the second soft anchor is a tubular sheath with a closed loop.

4. The tissue repair system of claim 3, wherein the closed loop is formed of round suture or flat tape.

5. The tissue repair system of claim 3, wherein the second soft anchor is a one-piece unitary construct.

6. The tissue repair system of claim 3, wherein the flexible coupler of the first soft anchor passes through the closed loop of the second soft anchor and back through the soft anchor sheath of the first soft anchor to lock the tissue repair system.

7. A tissue repair system, comprising:
a first soft anchor with a soft anchor sheath and at least one flexible strand extending through the soft anchor sheath; and
a second soft anchor knotlessly connected to the first soft anchor by the at least one flexible strand, wherein at least one of the first soft anchor and the second soft anchor is an all-suture soft anchor.

8. The tissue repair system of claim 7, consisting essentially of the first soft anchor connected to the second soft anchor.

9. The tissue repair system of claim 7, wherein the second soft anchor is a tubular sheath and wherein the at least one flexible strand of the first soft anchor passes through at least a portion of the tubular sheath of the second soft anchor and back through a soft anchor sheath of the first soft anchor to lock the tissue repair system.

10. The tissue repair system of claim 7, wherein the second soft anchor is a tubular sheath with a closed loop and wherein the at least one flexible strand of the first soft anchor passes through the closed loop of the second soft anchor and back through a soft anchor sheath of the first soft anchor to lock the tissue repair system.

11. The tissue repair system of claim 10, wherein the closed loop is formed of round, flat, or bifurcated suture.

12. The tissue repair system of claim 10, wherein the closed loop is configured to slide through the tubular sheath of the second soft anchor.

13. The tissue repair system of claim 7, wherein the first soft anchor and the second soft anchor reside in bone, and the at least one flexible strand forms at least one loop around, through, or over soft tissue to be attached to the bone.

14. A method of knotless tissue repair, comprising knotlessly connecting a first soft anchor to a second soft anchor with a single flexible coupler.

15. The method of claim 14, wherein the first soft anchor includes a first tubular sheath with the single flexible coupler extending through at least a portion of the first tubular sheath, wherein the second soft anchor includes a second tubular sheath, and wherein the method further comprises:
passing the single flexible coupler through, around, or over soft tissue to be attached to bone;
passing the single flexible coupler through at least a portion of the second tubular sheath or through a closed loop attached to the second tubular sheath; and
subsequently, passing the single flexible coupler back through the first tubular sheath.

16. The method of claim 15, wherein the first soft anchor further includes a shuttle/pull device and wherein the method further comprises employing the shuttle/pull device to pass the single flexible coupler back through the first tubular sheath and to form at least one knotless, closed, continuous loop around, through, or over the soft tissue.

17. The method of claim 16, further comprising:
passing the single flexible coupler through an eyelet of the shuttle/pull device; and
pulling the shuttle/pull device out of the first soft anchor to allow passing of the single flexible coupler through the first tubular sheath and to form the at least one knotless, closed, continuous loop around the soft tissue.

18. The method of claim 16, wherein the first soft anchor and the second soft anchor are secured in the bone, and wherein the soft tissue is tendon or ligament.

19. The method of claim 14, wherein each of the first soft anchor and the second soft anchor is an all-suture anchor formed of suture material.

20. The method of claim 14 consisting essentially of knotlessly connecting the first soft anchor to the second soft anchor with the single flexible coupler.
